(19) United States
(12) Patent Application Publication
(10) Pub. No.: US 2024/0225739 A1
(43) Pub. Date: Jul. 11, 2024

(54) SYSTEMS AND METHODS FOR RECONSTRUCTING AN ANATOMICAL STRUCTURE MODEL

(71) Applicants: Ohio State Innovation Foundation, Columbus, OH (US); Georgia Tech Research Corporation, Atlanta, GA (US)

(72) Inventors: Lakshmi Prasad Dasi, Dublin, OH (US); Huang Chen, Athens, GA (US); Beom Jun Lee, Atlanta, GA (US); Sri Krishna Sivakumar, Dublin, OH (US); Breandan Andre Butler Yeats, Atlanta, GA (US)

(21) Appl. No.: 18/560,134

(22) PCT Filed: May 11, 2022
(86) PCT No.: PCT/US2022/072240

§ 371 Date: Nov. 10, 2023

Related U.S. Application Data
(60) Provisional application No. 63/187,052, filed on May 11, 2021.

(51) Int. Cl. A61B 34/10; A61F 2/24; G06F 30/20

(52) U.S. Cl. CPC A61B 34/10; A61F 2/24; G06F 30/20; A61B 2034/105; A61B 2034/108; A61F 2240/001

(57) ABSTRACT

A heart valve model includes a parametric leaflet model representing geometries of a heart valve constructed based on at least one landmark point associated with the heart valve. The heart valve model includes a ray casted anatomical structure constructed based on an automatic aortic root reconstruction algorithm to extract a shape of an aorta. The heart valve also includes an additional anatomical structure, wherein the parametric leaflet model, the ray casted anatomical structure, and the additional anatomical structure are combined to obtain the heart valve model.

### PRIORITY

This application claims priority to U.S. Provisional Patent Application No. 63/187,052, filed May 11, 2021, which is incorporated by reference herein in its entirety.

### BACKGROUND

As an alternative approach to the highly invasive surgical aortic valve replacement (SAVR), transcatheter aortic valve replacement (TAVR) has become more prevalent in recent years. Unlike SAVR, whose size and implantation location can be precisely determined during surgery, TAVR relies on more thorough pre-surgery evaluations to determine the valve type, size, and deployment strategies with the help of advanced medical imaging techniques. A sub-optimal valve deployment has been associated with various adverse effects including elevated transvalvular pressure gradient, paravalvular leakage, aortic root rupture during implantation, coronary obstructions, etc.

To minimize these adverse effects, pre-TAVR evaluations may involve accurately reconstructing the patient's anatomy, generating a computational mesh, and using it for Finite Element Analysis (FEA) or/and Computational Fluid Dynamics (CFD) simulations. These state-of-the-art approaches based on patient-specific geometries have proven to have high predictive capabilities for TAVR outcomes and to prevent severe adverse events from happening in high-risk patients. As the technology of TAVR evolves, it has gradually expanded to low-risk patients. A comprehensive pre-surgery evaluation will become more important for this cohort since the long-term durability of the treatment is a primary concern. The rate of adverse events has been inversely correlated with durability. However, conducting these evaluations requires reconstructing a patient's anatomy from CT or MRI data, which is a tedious and time-consuming manual (3-6 hours per case). The following in-silico studies require even more expertise in solid/fluid mechanics and numerical methods, preventing them from being performed routinely in a typical clinical setting.

### SUMMARY

An exemplary system and method are disclosed that can be used to (1) generate a parametric heart valve model (e.g., aortic valve model or mitral valve model) with a few manually selected landmarks; (2) rapid automatic reconstruct the aorta and aortic valve leaflet geometries of a patient (e.g., for clinical pre-TAVR evaluation or other pre-procedural planning evaluation); (3) output readily usable computational mesh (e.g., for pre-TAVR in-silico studies, such as evaluation for coronary obstruction, root rapture and predicting valve hemodynamics). The exemplary system and method may be used for patient-specific computational or 3D printing evaluations that require the input of the patient geometry.

In some aspects, the exemplary system and method is configured to provide an automatic method to reconstruct the patient-specific geometries and generate meshes for in-silico studies with just a few manual inputs (about 5-10 minutes per case). To start the mesh generation from CT data, a user selects several landmark points related to the aortic valve. A parametric leaflet model is used to represent the valve geometry, followed by an automatic aortic root reconstruction algorithm that can extract the shape of the aorta. Later, the coronary arteries and calcium deposits are obtained automatically using a region-growing algorithm. Finally, the exemplary system and method generates a mesh for computational studies by combining all the mesh components. A visual inspection and possible manual clean-up are performed to ensure the accuracy and quality of the mesh.

In one aspect, a heart valve model includes a parametric leaflet model representing geometries of a heart valve constructed based on at least one landmark point associated with the heart valve. The heart valve model includes a ray casted anatomical structure constructed based on an automatic aortic root reconstruction algorithm to extract a shape of an aorta. The heart valve also includes an additional anatomical structure, wherein the parametric leaflet model, the ray casted anatomical structure, and the additional anatomical structure are combined to obtain the heart valve model.

In one aspect, a heart valve model includes multi-point ray casting of an aorta.

In one aspect, an anatomical structure model includes a first anatomical structure including a parametric leaflet model representing the first anatomical structure constructed based on at least one landmark point associated with the first anatomical structure. The anatomical structure model includes a second anatomical structure including a ray casted second anatomical structure constructed based on an automatic anatomical structure reconstruction algorithm to extract geometries of the second anatomical structure. The anatomical structure model also includes a third anatomical structure. The first, second, and third anatomical structures are combined to generate the anatomical structure model.

### BRIEF DESCRIPTION OF THE DRAWINGS

FIG. 1 is a flow chart of a semi-automatic patient-specific mesh generation process.

FIGS. 2A-2E are illustrations of an auto segmentation process. FIG. 2A shows the locations of the thirteen landmark points. FIG. 2B shows the parametric leaflet model with landmarks.

FIG. 2C shows aortic root with parametric leaflets. FIG. 2D shows coronary arteries and calcium deposits. FIG. 2E shows the final mesh output.

FIGS. 3A-3C are illustrations of the parametric leaflet generation process. FIG. 3A shows a skeleton connecting the landmarks using second order polynomials. FIG. 3B shows multiple second-order polynomials fitted to represent the leaflet surface. FIG. 3C shows the final triangular mesh.

FIGS. 4A-4F show samples of the parametric leaflets. FIGS. 4A-4C show three individual leaflets and FIGS. 4D-4F show full aortic valve.

FIG. 5 shows sample comparisons between the parametric leaflets (light gray) and the original image slices.

FIGS. 6A-6D are illustrations of an aorta reconstruction process. FIG. 6A shows detected aortic wall using a single-point ray-casting algorithm. FIG. 6B shows detected aortic wall using a multi-point ray-casting algorithm, wherein the edge detection accuracy has improved dramatically compared to that in FIG. 6A. FIG. 6C shows samples of the aortic wall detection results. FIG. 6D shows the final aorta model and mesh.

FIG. 7 shows comparisons between automatic (left) and manual (right) segmentation results.

FIG. 8 shows an illustrative computer architecture for a computer system 200 capable of executing the software components that can use the output of the exemplary method described herein. The computer architecture shown in FIG. 8 illustrates an example computer system configuration, and the computer 200 can be utilized to execute any aspects of the components and/or modules presented herein described as executing on the analysis system or any components in communication therewith.

### DETAILED DESCRIPTION

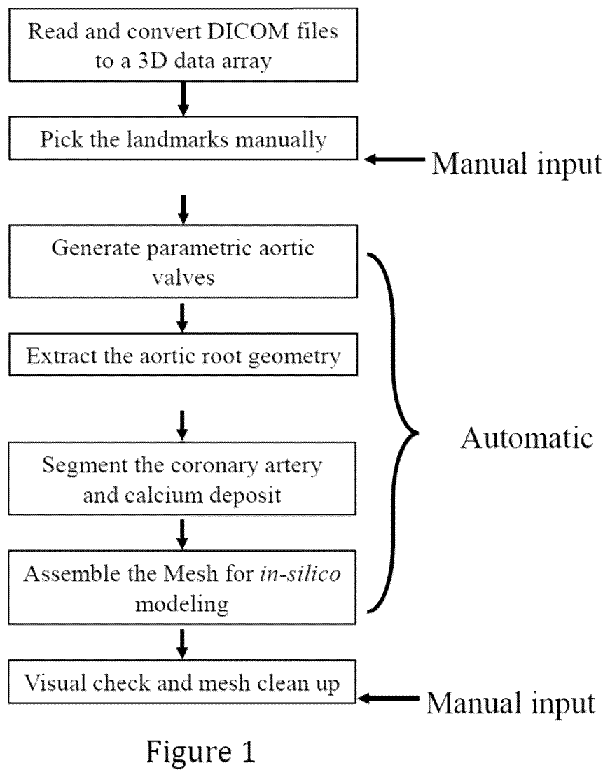

A flow chart of a semi-automatic mesh generation process is shown in FIG. 1. The algorithm consists of three main functions: (1) A parametric leaflet model, (2) a method to accurately determine the aortic root/aorta geometry, and (3) a method to assemble different components and output a final mesh for computational simulations.

In FIG. 1, to start the mesh generation from scanned data (e.g., CT data), a user (e.g., physician or technician) picks several landmark points related to the aortic valve. The exemplary system and method employ a parametric leaflet model to represent the valve geometry, which is then processed by an automatic aortic root reconstruction algorithm that can extract the shape of the aorta. Then, the exemplary system and method is configured to determine the coronary arteries and calcium deposits automatically using a region growing algorithm. Finally, the exemplary system and method generate a mesh for computational studies by combining all the mesh components. A visual inspection and surface manual clean-up are performed to ensure the accuracy and quality of the mesh.

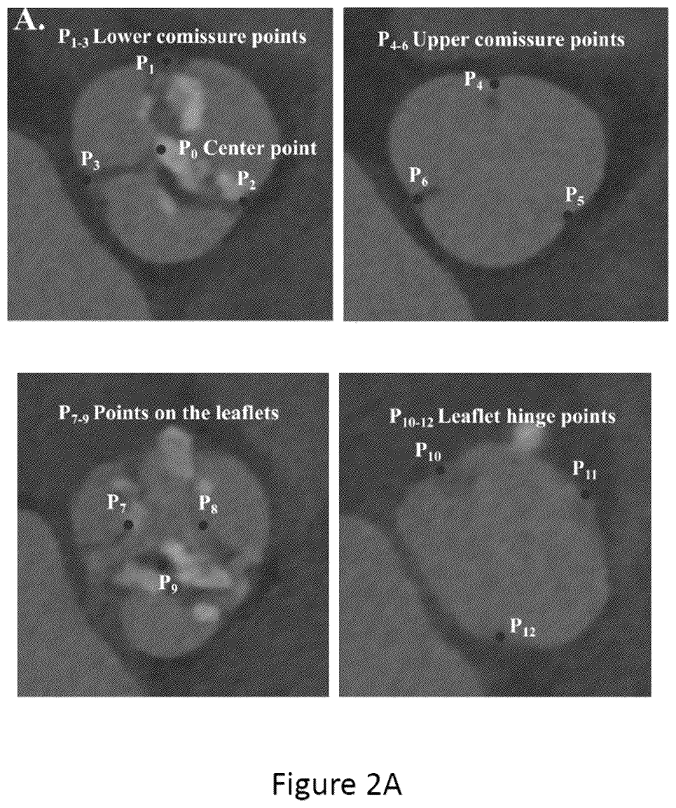

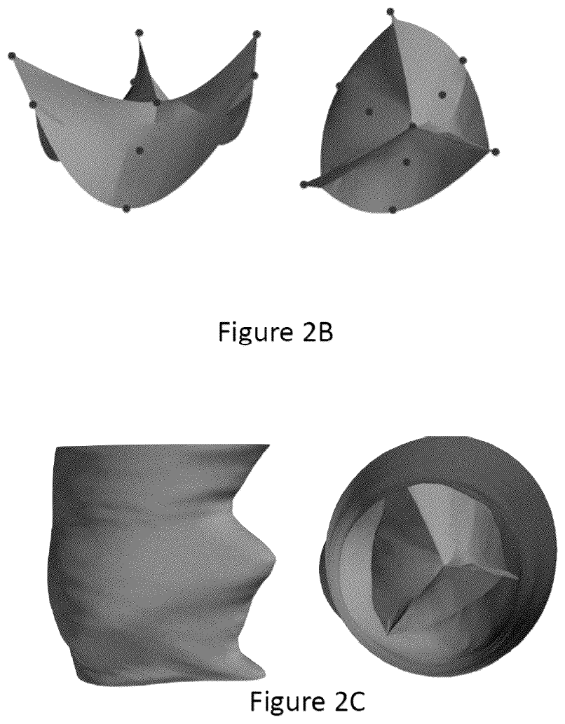

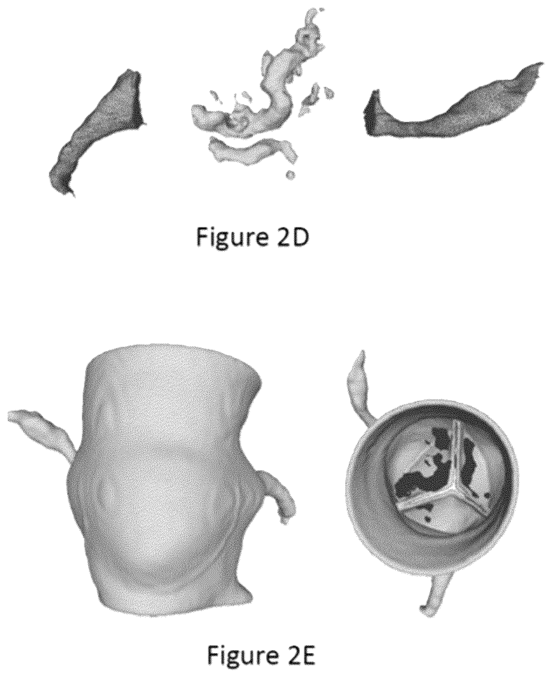

Since it is hard to identify the boundary of the aortic valve in CT scans, especially when there is little calcium deposit on the leaflets, a common practice during manual segmentation usually starts by segmenting the blood volume on the ventricular side at diastolic phases. The leaflets are subsequently generated by extruding the boundary of the blood pool. In the exemplary automatic segmentation process (FIG. 2), the method starts by selecting (e.g., by the user) landmark points in the 3D cardiac CT scans at the diastolic phase when the aortic valve is closed and the algorithm will take over and output the final mesh.

FIG. 2 shows a detailed illustration of an example auto segmentation process employed in the method of FIG. 1.

As shown in FIG. 2A, a total of 13 different landmarks are chosen for the parametric leaflet model. The points are: P₀ at the center coaptation point of the three leaflets; P₁₋₃ and P₄₋₆ are six commissural points. These two sets of points are aimed at resolving the finite coaptation height between the leaflets at the commissure. For example, P₁ and P₄ are at the lower and upper ends of this finite commissural coaptation line (FIG. 2B). P₁₋₉ are three surface points on the leaflets, while P₁₀₋₁₂ are the three leaflet hinges. The landmarks can be hand-picked by a clinician or can be obtained by artificial intelligence (A.I.) and/or machining learning approaches. The number and location of the landmarks are not limited to the described ones.

FIG. 2B shows the distributions of the landmark points in the parametric aortic valve model. With the guidance of these landmarks and the parametric leaflet model, an aortic root geometry is constructed in a slice-by-slice way using a multi-point ray-casting method described later.

FIG. 2C shows the reconstructed aortic root geometry with the parametric leaflets attached to it. Unlike many other parametric models, to ensure the output mesh quality in this invention, the leaflets are stitched to the aortic root to form a water-tight connection. Two extra landmarks at the coronary ostia are needed for auto segmenting the coronary arteries by an intensity-based region-growing method. Similarly, the calcium inside the aortic root domain is obtained by the same intensity-based segmentation method.

FIG. 2D shows the coronary artery and calcium geometries. The last step is to assemble the mesh to construct a computational mesh used in FEA/CFD simulations. Since the parametric leaflets and the aortic root are all surface meshes, specific thickness values are assigned to them based on physiological data. The three leaflets are separated by a finite distance comparable to the leaflet thickness to ensure their opening during numerical simulations. The final mesh is shown in FIG. 2E.

### Parametric Leaflet Model

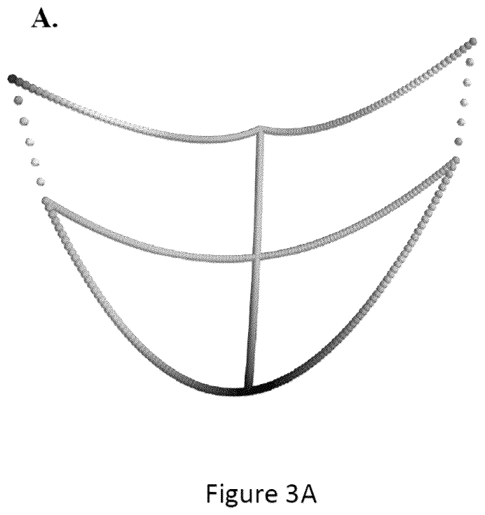

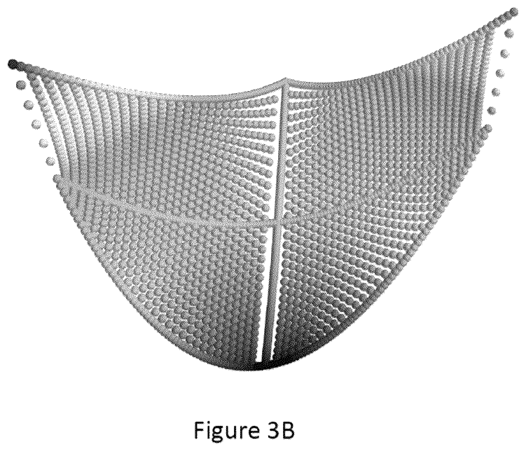

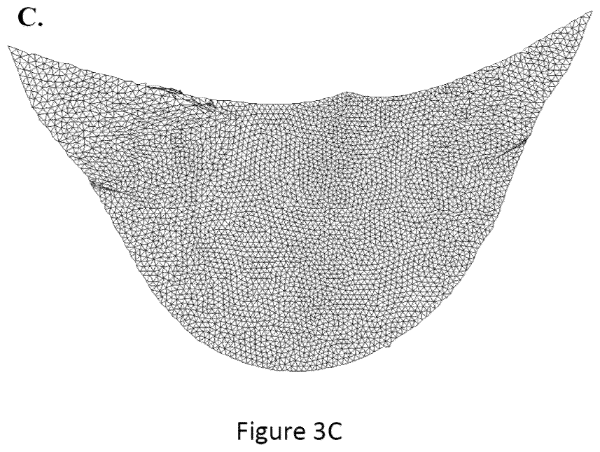

A parametric leaflet model maybe constructed based on selected landmarks (e.g., based on the thirteen landmark points in the example described above). There may be seven points on an individual leaflet surface (one center point, four commissural points, one point on the surface, and one hinge point). FIG. 3 shows an example parametric leaflet generation process of the method of FIG. 1.

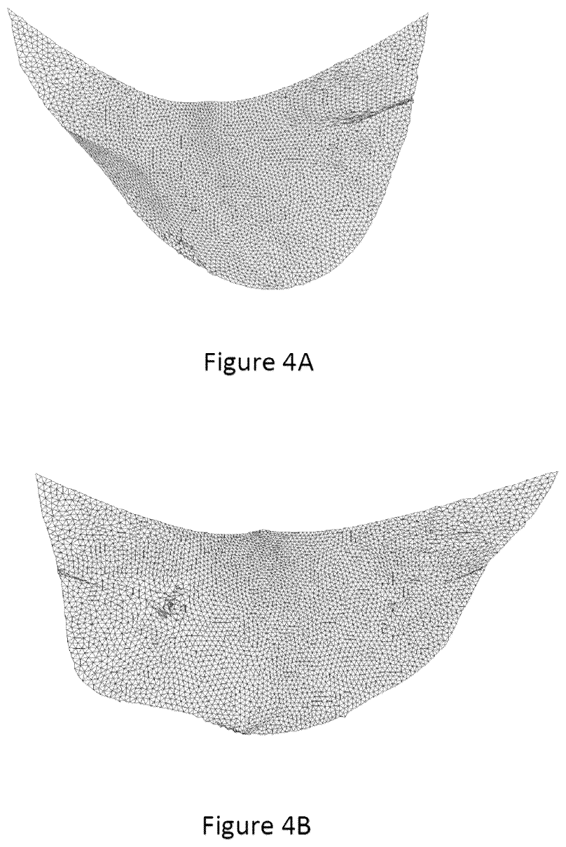

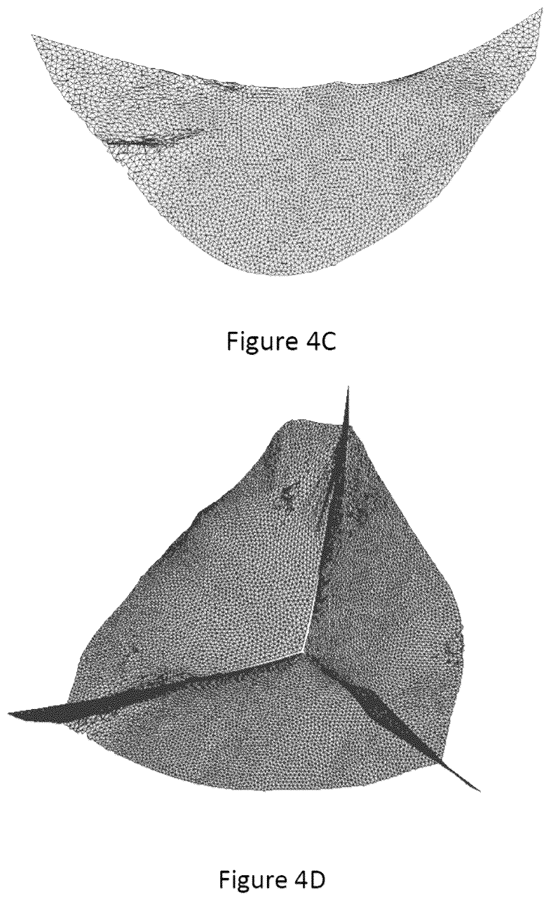

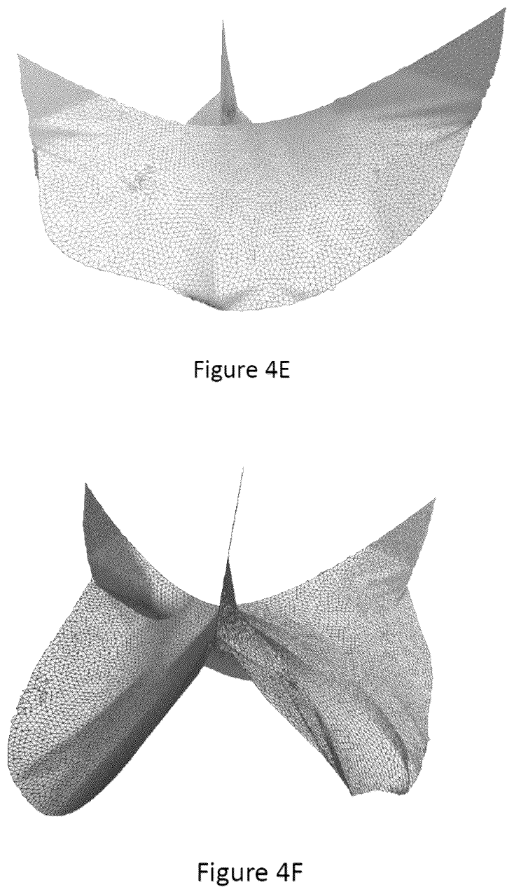

First, a leaflet skeleton is generated, in some aspects, using second-order polynomials connecting these points (FIG. 3A). Then, multiple second-order polynomials are fitted to the skeleton to create a surface (FIG. 3B). Finally, triangular meshes are used to represent the leaflet (FIG. 3C). Samples of the leaflet model are shown in FIG. 4. The way to generate the leaflet surface is not limited to the polynomial fit method described here. Other surface fitting method such as fitting by non-uniform Rational B-splines (NURBS) surfaces can be used with little or no modification to the procedures described here.

FIG. 4 shows example parametric leaflets models that can be generated in the method of FIG. 1. FIGS. 4A-4C show three individual leaflet meshes, and the corresponding aortic valve model is shown in FIGS. 4D-4E. This parametric leaflet model can handle complex surface geometries and capture an individual leaflet's irregular shape, as demonstrated in FIGS. 4A-4C.

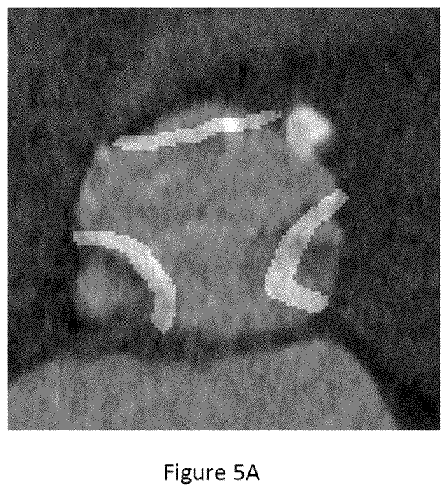

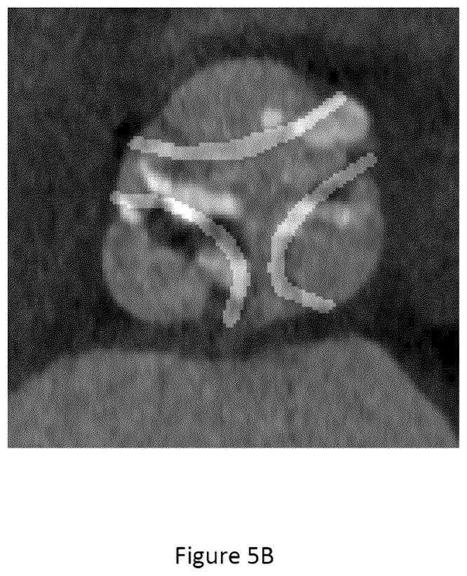

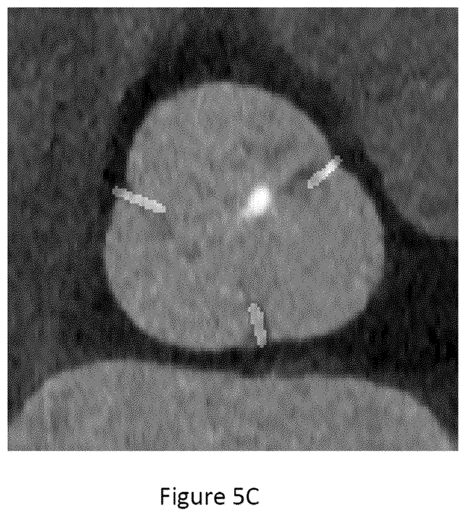

When the reconstructed leaflets are compared with the 3D CT image (FIG. 5), one can see that the parametric model (light gray) captures the original leaflet geometry very well. Some deviations from the original geometry are inevitable, but future improved algorithms will be able to locally deform the leaflet mesh to better match the original geometry.

### Aortic Root/Aorta Extraction Algorithm

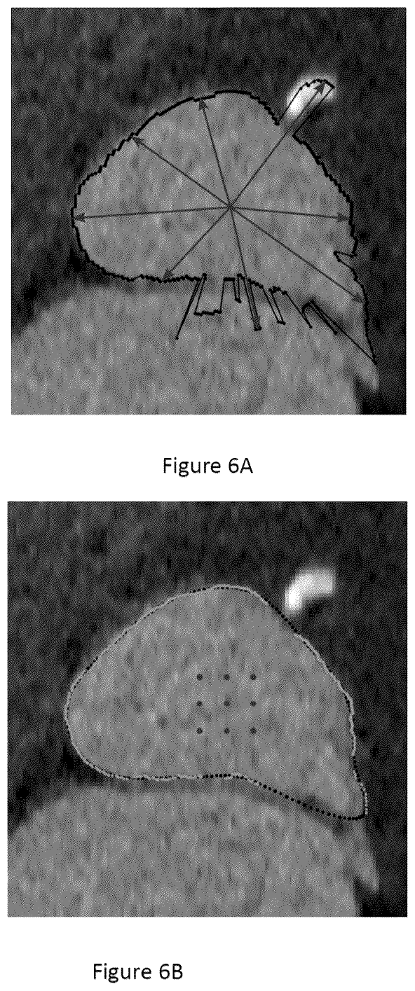

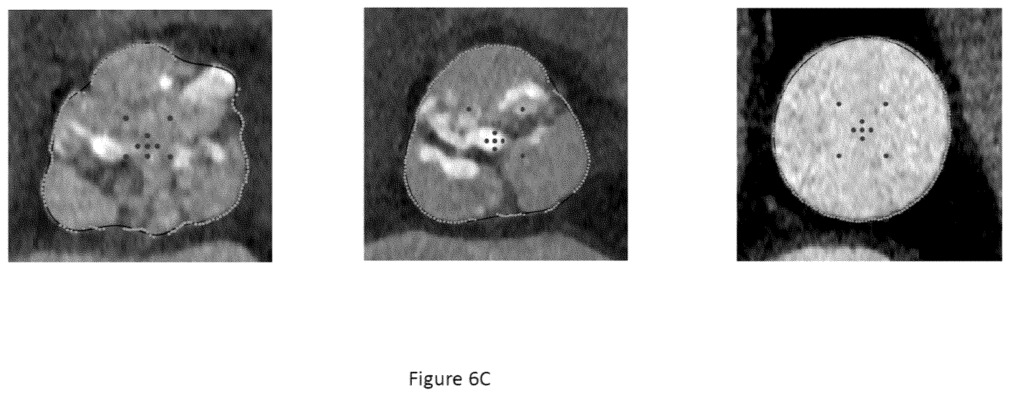

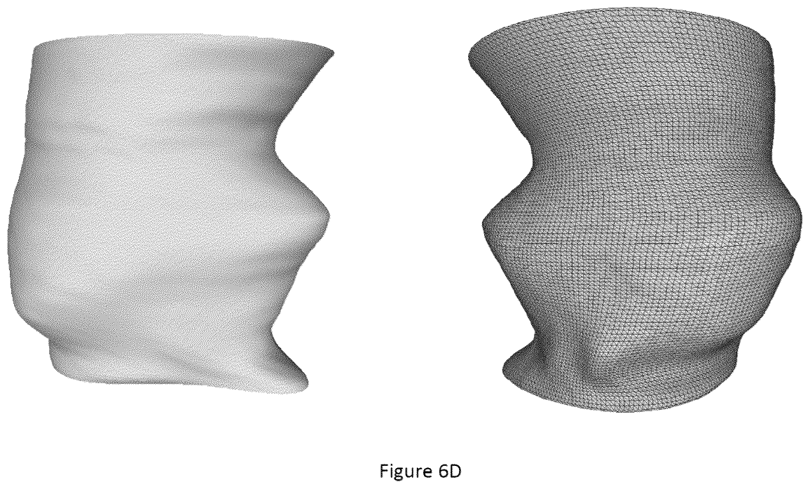

The extraction of the aortic root/aorta geometry may be based on an intensity-based, slice-by-slice, multi-point ray-casting edge detection algorithm (FIG. 6). FIG. 6 shows an example aorta reconstruction process of the method of FIG. 1.

After constructing the parametric leaflet model, the aortic annulus may be readily defined by the three leaflet hinge points. Slices parallel to the aortic annulus are extracted from the original 3D CT data. Starting with one point in the blood domain and casting rays in all directions, one can extract the intensity variations along the rays. A sudden jump of the intensity value from high (blood) to low (tissue) indicates the aortic wall (FIG. 6A). The contour of the aortic root in this slice is generated by connecting all the detected edge points. However, errors may occur during this edge detection process due to noise or poor image quality (FIG. 6A). To address this issue, a modified method that casts rays from a series of origins is introduced. Since most rays from different origins should converge at specific points on the actual aortic wall, and if there is a detection error for one ray, it is unlikely for rays from other origins to repeat the same error. Thus by comparing the detected edges from all origins, one can filter out the ambiguous points if they are too far away from the mean location. The mean value of the remaining points should accurately determine the aorta wall (FIG. 6B). If the points detected by different rays are too scattered at a specific location, this point will be discarded. FIGS. 6A and 6B have shown clearly that the multi-point approach can capture the aorta wall accurately. Finally, a cubic spline is fitted to all the detected points. In the sample slices shown in FIG. 3C, this algorithm demonstrates its ability to capture the aortic walls accurately from the left ventricular outflow graft to the ascending aorta. Note that the ray origins are different for all the slices (FIG. 3C). A five-point core and four satellite points derived from the neighboring slice's boundaries are used to improve the accuracy of edge detection. The contours from all the slices are stacked on top of each other to represent the wall of the aorta (FIG. 6D). The aorta geometry is smoothed by fitting a sixth-order polynomial to the points along the same longitude in the assembling process. The fitting method is not limited to polynomials. Other methods such as B-spline fit can be used as well. Triangle meshes are used to construct this geometry (FIG. 6D). Other surface mesh types such as rectangle or pentagon meshes can also be used.

### Coronary Artery and Calcium Deposit Segmentation

Both coronary arteries and calcium deposit geometries are segmented by an intensity-based region-growing algorithm. The coordinates of the two coronary ostia need to be specified (human input) as the starting points for the region growing algorithm. For the calcium, a global threshold of 850 Hounsfield Units (HU) is applied as suggested in, followed by a cleaning process that discards calcium blobs smaller than 20 voxels or located outside the aorta.

### Final Mesh Assembly

The final mesh assembling process is based on Boolean operations in the intensity space. First, the surface meshes of the aorta and the leaflets are thickened based on physiological values and voxelized. Later the coronary arteries and calcium deposits are added to the domain using Boolean operations. To prevent the leaflets from fusing, slits with the same width as the leaflet thickness are placed between them. Finally, a marching cubes algorithm converts the voxel data into an STL mesh. The assembled mesh will be visually inspected and corrected for mesh problems if there are any.

A Comparison with the Manual Segmentation Results

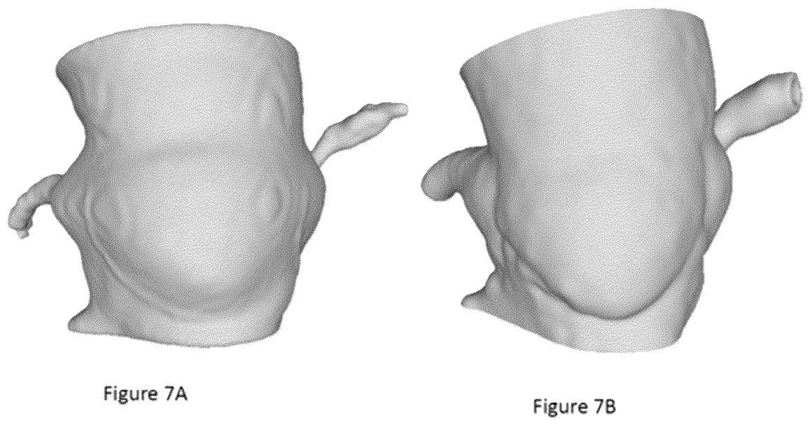

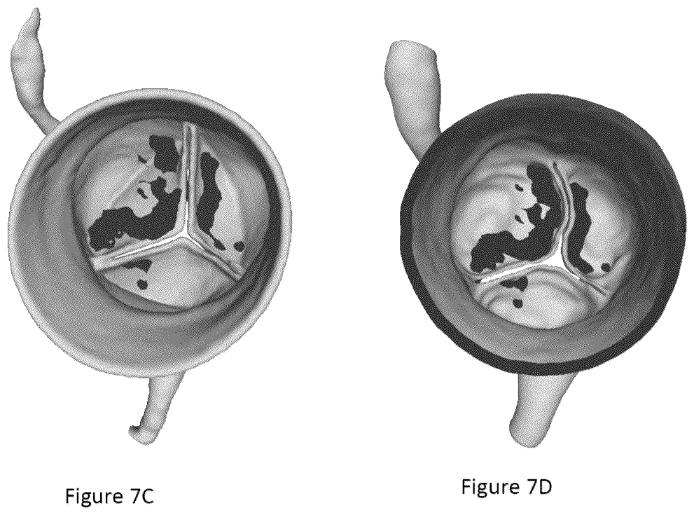

To ensure the accuracy of this method, comparisons with the manual segmentation results were carried out (FIG. 7). FIG. 7 shows a comparison between an example output of an automatic segmentation operation and a manual segmentation operation in the method of FIG. 1.

For the aorta, the auto-generated geometry (FIG. 7A) was very similar to that from the manual segmentation process (FIG. 7B). However, the manual result had more fine details due to less smoothing. In the en face views between the models (FIGS. 7C and 7D), they showed similar aortic leaflet geometries and calcium distributions. However, the current parametric leaflet model cannot resolve the curved coaptation line between the leaflets, since only three landmark points are used for this line. If an additional landmark point along the coaptation line is added or a local deformation algorithm is applied, the edge of the leaflet would be captured more accurately.

Although in exemplary system and method is discussed in relation to relation to aortic valve and TAVR, the exemplary system and method can be readily applied to the mitral valve and in other structural heart pre-procedural planning.

The exemplary system and method may be in daily clinical practices for TAVR and other structural heart presurgery evaluations. The exemplary system and method may be incorporated into software such as Materialise Mimics for users to construct patient-specific geometry with a few user input (i.e., user selected landmarks).

### DISCUSSION

Parametric heart valve models have been used in segmentation to address the issue of the time-consuming segmentation process in cases where the leaflets are hardly visible in medical images. Ionasec, R. et al. used parametric aorta and leaflet models to reconstruct both aortic and mitral valves from 4D cardiac CT and TEE. A learning-based algorithm was applied to the 4D images to identify and track landmarks throughout a cardiac cycle. A medial representation method was implemented by Pouch A.M. et al. to model the mitral valves from 3D echo images. They managed to capture the thickness of the mitral valve and its deformation during a cardiac cycle. The method has also been applied to reconstruct the anatomy of the aortic valve from 3D echo data (Pouch 2015). However, since the aortic valve leaflets has much higher contrast in the echo images, the authors did not mention whether this method could be applied to CT data as well. Lalys, F. et al. used a centerline detection approach to reconstruct the aorta followed by a registration algorithm to detect landmarks associated with the aortic valves. Later those landmarks were used to predict TAVR outcomes. Hosny, A. et al. used a similar approach described herein to generate parametric leaflet models for 3D printing. After printed together with manually segmented aorta and calcium, a model was built for valve sizing and pre-TAVR evaluation purposes. However, since 3D printing generally has a lower mesh quality requirement, their mesh could not be used in computational simulations directly. Unlike the work mentioned above, the exemplary system and method can generate a computational mesh directly from 3D CT data with just a few manual inputs.

It should be appreciated that the logical operations described above can be implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

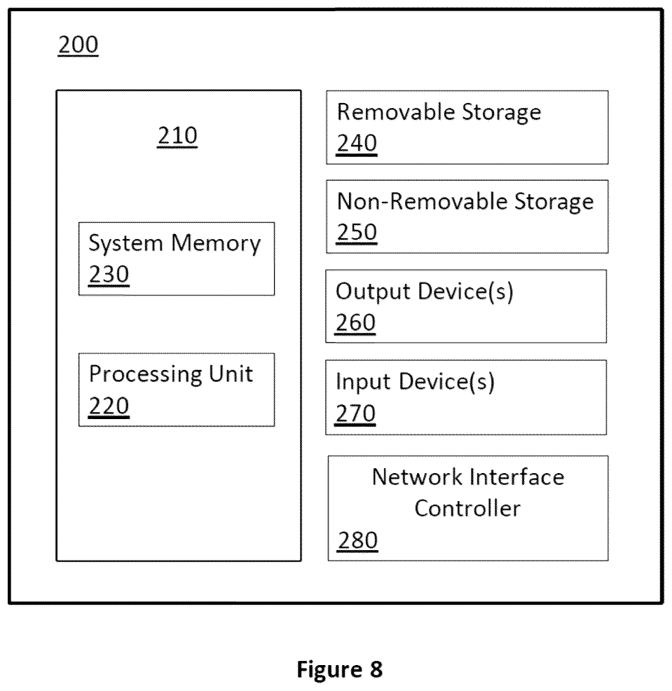

FIG. 8 shows an illustrative computer architecture for a computer system 200 capable of executing the software components that can use the output of the exemplary method described herein. The computer architecture shown in FIG. 8 illustrates an example computer system configuration, and the computer 200 can be utilized to execute any aspects of the components and/or modules presented herein described as executing on the analysis system or any components in communication therewith.

In an aspect, the computing device 200 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an aspect, virtualization software may be employed by the computing device 200 to provide the functionality of a number of servers that is not directly bound to the number of computers in the computing device 200. For example, virtualization software may provide twenty virtual servers on four physical computers. In an aspect, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing device 200 typically includes at least one processing unit 220 and system memory 230. Depending on the exact configuration and type of computing device, system memory 230 may be volatile (such as random-access memory (RAM)), non-volatile (such as read-only memory (ROM), flash memory, etc.), or some combination of the two.

This most basic configuration is illustrated in FIG. 8 by dashed line 210. The processing unit 220 may be a standard programmable processor that performs arithmetic and logic operations necessary for operation of the computing device 200. While only one processing unit 220 is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application specific circuits (ASICs). Thus, while instructions may be discussed as executed by a processor, the instructions may be executed simultaneously, serially, or otherwise executed by one or multiple processors. The computing device 200 may also include a bus or other communication mechanism for communicating information among various components of the computing device 200.

Computing device 200 may have additional features/functionality. For example, computing device 200 may include additional storage such as removable storage 240 and non-removable storage 250 including, but not limited to, magnetic or optical disks or tapes. Computing device 200 may also contain network connection(s) 280 that allow the device to communicate with other devices such as over the communication pathways described herein. The network connection(s) 280 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 200 may also have input device(s) 270 such as keyboards, keypads, switches, dials, mice, track balls, touch screens, voice recognizers, card readers, paper tape readers, or other well-known input devices. Output device(s) 260 such as printers, video monitors, liquid crystal displays (LCDs), touch screen displays, displays, speakers, etc. may also be included. The additional devices may be connected to the bus in order to facilitate communication of data among the components of the computing device 200. All these devices are well known in the art and need not be discussed at length here.

The processing unit 220 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 220 for execution. Example tangible, computer-readable media may include, but is not limited to, volatile media, non-volatile media, removable media and non-removable media implemented in any method or technology for storage of information such as computer readable instructions, data structures, program modules or other data. System memory 230, removable storage 240, and non-removable storage 250 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture 200 in order to store and execute the software components presented herein. It also should be appreciated that the computer architecture 200 may include other types of computing devices, including hand-held computers, embedded computer systems, personal digital assistants, and other types of computing devices known to those skilled in the art. It is also contemplated that the computer architecture 200 may not include all of the components shown in FIG. 8, may include other components that are not explicitly shown in FIG. 8, or may utilize an architecture different than that shown in FIG. 8.

In an example implementation, the processing unit 220 may execute program code stored in the system memory 230. For example, the bus may carry data to the system memory 230, from which the processing unit 220 receives and executes instructions. The data received by the system memory 230 may optionally be stored on the removable storage 240 or the non-removable storage 250 before or after execution by the processing unit 220.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Moreover, the various components may be in communication via wireless and/or hardwire or other desirable and available communication means, systems and hardware. Moreover, various components and modules may be substituted with other modules or components that provide similar functions.

The computer architecture 200 includes software and/or hardware components and modules needed to enable the function of the modeling, simulation, and methods disclosed in the present disclosure. In some aspects, the computer architecture 200 may include artificial intelligence (A.I.) modules or algorithms and/or machine learning (M.L.) modules or algorithms (e.g., stored in the system memory 230, removable storage 240, non-removable storage 250, and/or a cloud database). The A.I. and/or M.L. modules/algorithms may improve the predictive power of the models, simulations, and/or methods disclosed in the present disclosure. For example, by using a deep learning, A.I., and/or M.L. model training including patient information and any relevant input data to the computational model, the predictive power of the computational model may be greatly enhanced. The A.I. and/or M.I. modules/algorithms also help improving sensitivity and specificity of the prediction as the database grows. In some aspects, the computer architecture 200 may include virtual reality (VR), augmented reality (AR) and/or mixed reality display(s), headset(s), glass(es), or any other suitable display device(s) as a part of the output device(s) 260 and/or the input device(s) 270. In some aspects, the display device(s) may be interactive to allow an user to select from options including with or without AR, with or without VR, or fused with real time clinical imaging to help clinician interact and make decisions.

Although example aspects of the present disclosure are explained in some instances in detail herein, it is to be understood that other aspects are contemplated. Accordingly, it is not intended that the present disclosure be limited in its scope to the details of construction and arrangement of components set forth in the following description or illustrated in the drawings. The present disclosure is capable of other aspects and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary aspects include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example aspects, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified. As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The exemplary system and method can significantly reduce the manual effort needed to build the patient-specific model from several hours to only a few minutes. It will be apparent to those skilled in the art that various modifications and variations can be made in the present disclosure without departing from the scope or spirit of the invention. Other aspects of the disclosure will be apparent to those skilled in the art from consideration of the specification and practice of the methods disclosed herein. It is intended that the specification and examples be considered as exemplary only, with a true scope and spirit of the invention being indicated by the following claims.

1. A heart valve model comprising:
a parametric leaflet model representing geometries of a heart valve constructed based on at least one landmark point associated with the heart valve;
a ray casted anatomical structure constructed based on an automatic aortic root reconstruction algorithm to extract a shape of an aorta; and
an additional anatomical structure, wherein the parametric leaflet model, the ray casted anatomical structure, and the additional anatomical structure are combined to obtain the heart valve model.

2. The heart valve model of claim 1, wherein the at least one landmark point is any distinguishable anatomical feature.

3. The heart valve model of claim 1, wherein the at least one landmark point is obtained based on a CT scan.

4. The heart valve model of claim 1, wherein the at least one landmark point is based on an MRI scan.

5. The heart valve model of claim 1, wherein the at least one landmark point is based on an ultra-sound scan.

6. The heart valve model of claim 1, wherein the at least one landmark point is patient specific.

7. The heart valve model of claim 1, wherein the automatic aortic root reconstruction algorithm comprises an intensity-based, slice-by-slice, multi-point ray-casting edge detection algorithm.

8. The heart valve model of claim 7, wherein the automatic aortic root reconstruction algorithm compares detected edges from all origins to capture an aorta wall edge.

9. The heart valve model of claim 1, wherein the additional anatomical structure comprises geometries of a coronary artery and calcium deposits.

10. The heart valve model of claim 9, wherein the geometries of the coronary artery and the calcium deposits are segmented by an intensity-based region-growing algorithm.

11. The heart valve model of claim 1, wherein the parametric leaflet model, the ray casted anatomical structure, and the additional anatomical structure are combined to obtain a geometric mesh file suitable for computational modeling.

12. A heart valve model comprising multi-point ray casting of an aorta.

13. The heart valve model of claim 12, wherein the heart valve model further comprises a combination of the multi-point ray casting and a parametric leaflet model representing geometries of a heart valve.

14. The heart valve model of claim 12, further comprising geometries of calcium deposits and a coronary artery.

15. The heart valve model of claim 14, wherein the geometries of the calcium deposits and the coronary artery are segmented by an intensity-based region-growing algorithm.

16. An anatomical structure model comprising:
a first anatomical structure comprising a parametric leaflet model representing the first anatomical structure constructed based on at least one landmark point associated with the first anatomical structure;
a second anatomical structure comprising a ray casted second anatomical structure constructed based on an automatic anatomical structure reconstruction algorithm to extract geometries of the second anatomical structure; and
a third anatomical structure, wherein the first, second, and third anatomical structures are combined to generate the anatomical structure model.

17. The anatomical structural model of claim 16, wherein the at least one landmark point is obtained using a CT scan, an MRI scan, or an ultra-sound scan.

18. The anatomical structural model of claim 16, wherein the at least one landmark point is patient specific.

19. The anatomical structural model of claim 16, wherein the parametric leaflet model is based on at least seven or more landmark points.

20. The anatomical structural model of claim 16, wherein the third anatomical structure comprises a coronary artery or calcium deposits.
